(19) United States
(12) Patent Application Publication
(10) Pub. No.: US 2024/0016761 A1
(43) Pub. Date: Jan. 18, 2024

(54) NUTRITIONAL COMPOSITION FOR INHIBITING SURVIVAL OF RNA VIRUS AND METHOD FOR TREATING RNA VIRUS INFECTION

(71) Applicant: Trade Wind Biotech Co. Ltd., TaipeiCity (TW)

(72) Inventors: Jui-Jen Chang, Taipei City (TW); Yeh Chen, Taichung City (TW)

(21) Appl. No.: 18/350,278

(22) Filed: Jul. 11, 2023

Related U.S. Application Data
(60) Provisional application No. 63/368,253, filed on Jul. 12, 2022.

(51) Int. Cl. A61K 31/05; A61K 31/351; A61K 31/726; A61P 31/14; A23L 33/00

(52) U.S. Cl. CPC A61K 31/05; A61K 31/351; A61K 31/726; A61P 31/14; A23L 33/30

(57) ABSTRACT

The present disclosure relates to a nutritional composition for inhibiting survival of an RNA virus and a method for treating an RNA virus infection. The nutritional composition consists of an astaxanthin extract and at least one probiotic extract. The at least one probiotic extract is selected from the group consisting of a lipoteichoic acid and a peptidoglycan. The method for treating the RNA virus infection includes administering an effective concentration of a nutritional composition to a subject in need thereof, wherein the nutritional composition consists of an astaxanthin extract and at least one probiotic extract, and the at least one probiotic extract is selected from the group consisting of a lipoteichoic acid and a peptidoglycan.

### RELATED APPLICATIONS

This application claims priority to U.S. Provisional Application No. 63/368,253, filed on Jul. 12, 2022, which is herein incorporated by reference.

### BACKGROUND

### Technical Field

The present disclosure relates to a nutritional composition and a method for treating a virus infection. More particularly, the present disclosure relates to a nutritional composition for inhibiting survival of an RNA virus and a method for treating an RNA virus infection.

### Description of Related Art

The rapid rise in the number of emerging pathogens in the world's population represents a serious global health problem and underscores the need to develop broad spectrum anti-infectives that target common components of large classes of pathogens. An RNA virus is a virus that has ribonucleic acid (RNA) as its genetic material. Notable human diseases caused by the RNA virus include the common cold, influenza, severe acute respiratory syndrome (SARS), Middle East respiratory syndrome (MERS), dengue fever, hepatitis C, hepatitis E, West Nile fever, Ebola virus disease, rabies, polio, mumps, and measles. The RNA virus has higher variability because of lack of error-correcting DNA polymerases.

For example, the worldwide spread of the coronavirus disease 2019 (COVID-19) pandemic caused by a novel severe acute respiratory syndrome coronavirus 2 (SARS-CoV-2) since late December 2019 has put people in devastating difficulty, with remarkable impacts on the global public health, economic systems, and habits of the entire world population. Despite some effective vaccines or drugs being approved and extensively administered, the long-term efficacy and safety of this intervention approach are constantly under debate as coronaviruses rapidly mutate, and several SARS-CoV-2 variants have already been identified around the world.

The new emerging variants of SARS-CoV-2, including Delta, Omicron and its sub-variants BA.2.12.1, BA.4, BA.5, have enhanced transmissibility, virulence and/or immune evasion characteristics and still posed threatens to global human beings. Current FDA-approved COVID-19 vaccines cannot totally prevent from SARS-CoV-2 infections. So people started to find alternative ways to increase their body's capability to deal with outside threats, such as exercising or taking nutraceuticals. Up to date, no proof-of-concept nutritional supplements that are developed for COVID-19 prevention.

Therefore, development of specific nutritional composition to boost people's immune system and protect people from infections caused by RNA viruses is urgently needed.

### SUMMARY

According to an aspect of the present disclosure, a nutritional composition for inhibiting survival of an RNA virus is provided. The nutritional composition consists of an astaxanthin extract and at least one probiotic extract, wherein the at least one probiotic extract is selected from the group consisting of a lipoteichoic acid and a peptidoglycan.

According to another aspect of the present disclosure, a method for treating an RNA virus infection includes administering an effective concentration of a nutritional composition to a subject in need thereof, wherein the nutritional composition consists of an astaxanthin extract and at least one probiotic extract, and the at least one probiotic extract is selected from the group consisting of a lipoteichoic acid and a peptidoglycan.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram of anti-viral mechanisms of a nutritional composition for inhibiting survival of an RNA virus according to one embodiment of the present disclosure.

FIG. 2 shows inhibition of a binding of a SARS-CoV-2 spike protein to a human angiotensin-converting enzyme 2 (ACE2) by an astaxanthin (ASTA) extract, a lipoteichoic acid (LTA), or a peptidoglycan (PGN).

FIG. 3 shows the dose-dependent effects of the ASTA extract, the LTA or the nutritional composition for inhibiting survival of the RNA virus of the present disclosure on an activity of a main protein (M^{pro}) of the SARS-CoV-2.

FIG. 4 shows inhibition of an activity of a papain-like protein (PL^{pro}) of the SARS-CoV-2 by the ASTA extract, the LTA or the PGN.

FIG. 5 shows the inhibitory effect on an infection efficiency of a SARS-CoV-2 variant (Omicron) of the ASTA extract, the LTA, the PGN or the nutritional composition for inhibiting survival of the RNA virus of the present disclosure.

FIG. 6A is a schematic view showing a protein structure of an RNA-dependent RNA polymerase (RdRp) of an influenza A virus.

FIG. 6B is a schematic view showing a protein structure of an RdRp of the SARS-CoV-2.

FIG. 7A shows inhibition of an activity of the RdRp of the influenza A virus by the ASTA extract.

FIG. 7B shows inhibition of an activity of the RdRp of the SARS-CoV-2 by the ASTA extract.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

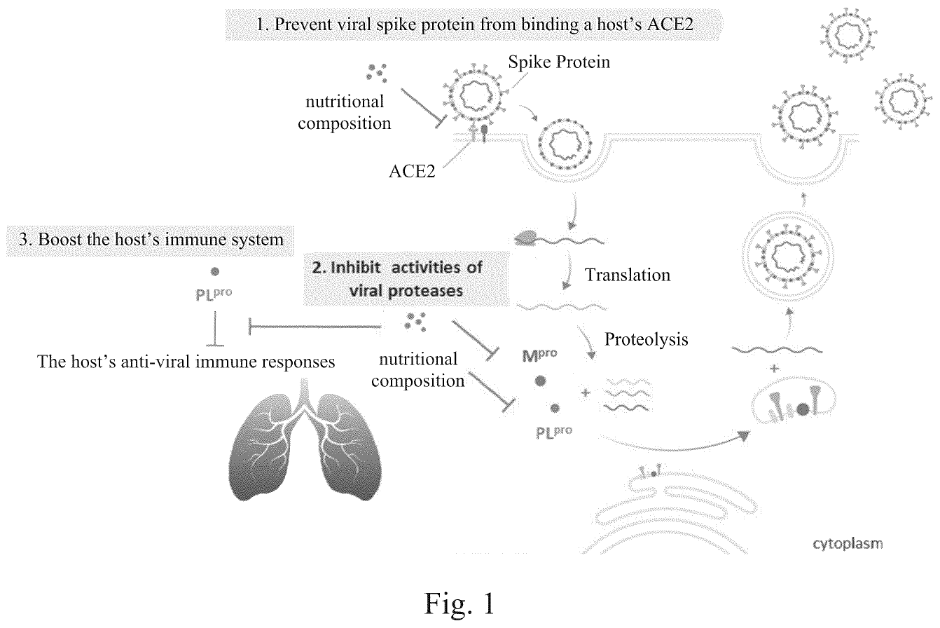

Please refer to FIG. 1, which is a schematic diagram of anti-viral mechanisms of a nutritional composition for inhibiting survival of an RNA virus according to one embodiment of the present disclosure. Reasons why the COVID-19 pandemic can quickly sweep the world are due to its rapid inflection rate, strong replication ability after entering a host's body, and adaptability to the host's immune system. An infection ability of a severe acute respiratory syndrome coronavirus 2 (SARS-CoV-2) comes from a binding of spike proteins on a surface of the SARS-CoV-2 to an angiotensin-converting enzyme 2 (ACE2), and the further replication and survival wrestling with the host's immune system are contributed to a main protease (M^{pro}) and a papain-like protease (PL^{pro}) The M^{pro} will be expressed after the SARS-CoV-2 enters a host, and main function of the M^{pro} is to cleave two long polyproteins expressed by SARS-CoV-2 replicase genes, so as to perform viral nucleic acid replication and transcription. It should be noted that, though a sequence similarity of the M^{pro} in different coronaviruses is not high, there is a highly retained three-dimensional structure in the substrate-binding pocket of the M^{pro}. The PL^{pro} is a multifunctional enzyme, not only is responsible for processing the viral polypeptide into functional units but also take part in negative-regulating the immune responses of the host, such as functions of deubiquitinating (DUB) and delSGylating (delSG) activity. Therefore, the M^{pro} and the PL^{pro} have been important targets for the long-term treatment strategy of COVID-19.

According to one aspect of the present disclosure, the nutritional composition for inhibiting survival of the RNA virus is provided. The nutritional composition for inhibiting survival of the RNA virus of the present disclosure consists of an astaxanthin (ASTA) extract and at least one probiotics extract, wherein the at least one probiotic extract is selected from the group consisting of a lipoteichoic acid (LTA) and a peptidoglycan (PGN). Particularly, an effective concentration of the ASTA extract can be equal to or larger than 0.1 wt %, an effective concentration of the LTA can be equal to or larger than 1 mg/ml, and an effective concentration of the PGN can be equal to or larger than 1 mg/ml. The RNA virus can be the SARS-CoV-2 or an influenza A virus. Moreover, the LTA and the PGN can be extracted from a probiotic bacterium. In detail, the ASTA extract can be an astaxanthin (ASTA) dissolved in a solvent, the solvent can be ethanol, methanol, acetone, dimethyl sulfoxide (DMSO), a mixture of DMSO and acetone (1:1), chloroform or dichloromethane. The solubility of the ASTA in ethanol is 0.038 g/L, the solubility of the ASTA in methanol is 0.04 g/L, the solubility of the ASTA in acetone is 0.55 g/L, the solubility of the ASTA in DMSO is 0.5 g/L, the solubility of the ASTA in the mixture of DMSO and acetone is 2.03 g/L, the solubility of the ASTA in chloroform is 10 g/L, and the solubility of the ASTA in dichloromethane is 30 g/L.

Specifically, the ASTA is originally used as a supplement to provide pink color in salmonid and crustacean aquaculture for over two decades. Recent researches have identified several health benefits of the ASTA, including extraordinary antioxidant effect, anti-inflammation, anti-diabetic activity, anti-cancer activity, cardiovascular disease prevention and immuno-modulation functions. The LTA and the PGN, which can be extracted from the probiotic bacterium in general, have also been described hi several literatures, including modulation of the host's immune system, balancing the gut microbiomes, reduction of the respiratory tracts infections by influencing the gut-lung axis.

According to another aspect of the present disclosure, a method for treating an RNA virus infection includes administering an effective concentration of a nutritional composition to a subject in need thereof, wherein the nutritional composition consists of the ASTA extract and at least one probiotic extract, and the at least one probiotic extract is selected from the group consisting of the LTA acid and the PGN. The nutritional composition can be capable as an RNA-dependent RNA polymerase (RdRp) inhibitor. In addition, the RNA virus infection can be caused by the influenza A virus or can be caused by the SARS-CoV-2. The nutritional composition can be capable of inhibiting a binding of a SARS-CoV-2 spike protein to an angiotensin-converting enzyme 2 (ACE2). The nutritional composition can be capable of inhibiting an activity of the M^{pro} of the SARS-CoV-2. The nutritional composition also can be capable of inhibiting an activity of the PL^{pro} of the SARS-CoV-2. Particularly, an effective concentration of the ASTA extract can be equal to or larger than 0.1 wt %, an effective concentration of the LTA can be equal to or larger than 1 mg/ml, and an effective concentration of the PGN can be equal to or larger than 1 mg/ml.

Relying on the excellent and diverse bioactivities mention-above, the nutritional composition for inhibiting survival of the RNA virus of the present disclosure can be considered as a very up-and-coming candidate in combating the RNA virus infection, such as COVID-19 and avian influenza.

1. The Inhibitory Effect on the Binding of the SARS-CoV-2 Spike Protein to a Human ACE2

The inhibitory effect of the ASTA extract, the LTA and the PGN on an interaction between the SARS-CoV-2 spike protein and the human ACE2 are measured by a SARS-CoV-2 ELISA Kit. Briefly, horseradish peroxidase (HRP)-conjugate-ACE2 is pre-incubated with 0.2 wt % ASTA extract, 5 mg/ml LTA or 1 mg/ml PGN at room temperature for 30 minutes, followed by addition to an ELISA plate pre-coated with a receptor-binding domain of the SARS-CoV-2 spike protein at 37° C. for 1 hour. The ASTA extract is prepared by dissolving the ASTA in ethanol. In addition, horseradish peroxidase (HRP)-conjugate-ACE2 is pre-incubated with positive control mAb or negative control mAb as a positive control group or a negative control group, respectively. Then, 3,3′,5,5′-tetramethylbenzidine (TMB) substrate solution is added to each well and incubated at 37° C. for 20 minutes to detect a HRP activity. A color development is stopped and intensity is determined at OD450.

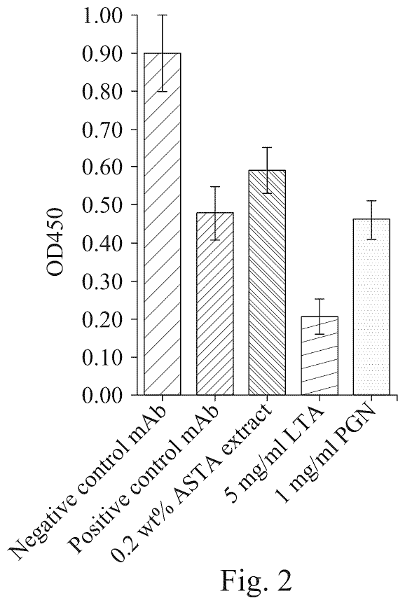

Please refer to FIG. 2, which shows inhibition of a binding of the SARS-CoV-2 spike protein to a human angiotensin-converting enzyme 2 (ACE2) by the ASTA extract, the LTA, or the PGN. In FIG. 2, the inhibitory effect on the binding of the SARS-CoV-2 spike protein to the human ACE2 of the test group treated with 1 mg/ml PGN is close to but a bit lower than that of the positive control group, the inhibitory effect on the binding of the SARS-CoV-2 spike protein to the human ACE2 of the test group treated with 0.2 wt % ASTA extract is slightly higher than that of the positive control group, and the inhibitory effect on the binding of the SARS-CoV-2 spike protein to the human ACE2 of the test group treated with 5 mg/ml LTA is the most significant among the three test groups and the positive control group. The results in FIG. 2 indicate that ingredients of the nutritional composition for inhibiting survival of the RNA virus of the present disclosure can effectively inhibit the binding of SARS-CoV-2 spike protein to the human ACE2.

2. The Inhibitory Effect on the Activity of the M^{pro} of the SARS-CoV-2

The inhibitory effect of the ASTA extract and the LTA on the activity of the M^{pro} of the SARS-CoV-2 is assessed by FRET based assay described as follows. Briefly, a purified M^{pro} of the SARS-CoV-2 is first pre-incubated with various concentrations of the ASTA extract (0.4 wt %, 0.2 wt % and 0.1 wt %), a vitamin C (0.2 wt % and 0.1 wt % for comparison), the LTA (1 mg/ml, and 2 mg/ml), or Example 1 of the nutritional composition for inhibiting survival of the RNA virus of the present disclosure (hereafter referred to as “Example 1”) including 1 mg/ml LTA and 0.1 wt % ASTA extract in an assay buffer (20 mM Tris 7.8, 20 mM NaCl) at room temperature for 30 minutes, respectively. A fluorescent protein substrate of the M^{pro} of the SARS-CoV-2 is then added to initiate the reaction. A fluorescence signal is monitored at an emission wavelength of 474 nm with an excitation wavelength at 434 nm using Synergy™ H1 hybrid multi-mode microplate reader (BioTek Instruments, Inc.). Data are performed with two technical replicates.

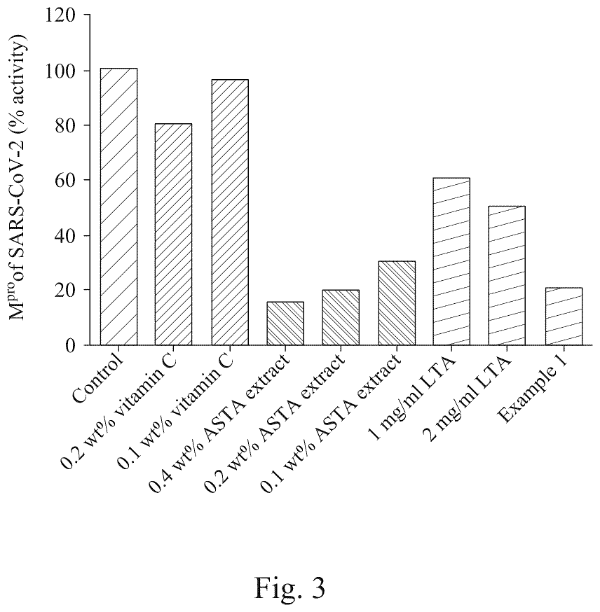

Pease refer to FIG. 3, which shows the dose-dependent effects of the ASTA extract, the LTA or Example 1 on an activity of the M^{pro} of the SARS-CoV-2, In FIG. 3, the inhibitory effect on the activity of the M^{pro} of the SARS-CoV-2 of a comparative group treated with 0.1 vitamin C is pretty close to but still slightly better than that of a control group. However, the inhibitory effect of a comparative group treated with 0.2 wt % vitamin C only reaches 20%, In contrast, the results in test groups treated with the LTA and/or the ASTA extract show more significant inhibitory effect on the activity of the M^{pro} of the SARS-CoV-2, especially the test groups treated with the ASTA extract. The inhibitory effect of the LTA and the ASTA extract are dose-dependent. Particularly, the activity of the M^{pro} of the SARS-CoV-2 in the test groups treated with 0.1 wt % ASTA extract, 0.2 wt % ASTA extract and 0.4 wt % ASTA extract are decreased to dose to 30%, close to 20% and below 20%, respectively. The activity of the M^{pro} of the SARS-CoV-2 in the test groups treated with 1 mg/ml LTA and 2 mg/ml LTA is decreased to close to 60% and close to 50%, respectively. It should be noted that, the inhibitory effect on the activity of the M^{pro} of the SARS-CoV-2 of the test group treated with Example 1 is comparable with that of the test group treated with 0.2 wt % ASTA extract, and also greater than either that of the test group treated with 1 mg/ml LTA or that of the test group treated with 0.1 wt % ASTA extract. That is, the results in the FIG. 3 not only show that the nutritional composition for inhibiting survival of the RNA virus of the present disclosure can effectively inhibit the activity of the M^{pro} of the SARS-CoV-2, but also indicate a possible synergism on inhibition of the activity of the M^{pro} of the SARS-CoV-2.

3. The Inhibitory Effect on the Activity of the PL^{pro} of the SARS-CoV-2

To access the inhibitory effect of the ASTA extract, the LTA or the PGN on the activity of the PL^{pro} of the SARS-CoV-2, a recombinant PL^{pro} is incubated with 0.2 wt % ASTA extract, 17.5 mg/ml LTA, or 1 mg/ml PGN at room temperature for 30 minutes. Moreover, the recombinant PL^{pro} is incubated with DMSO as a control group, wherein a final concentration of the DMSO is 10% v/v, A peptide substrate (Z-RLRGG-AMC) is then added to start the reaction. A fluorescence signal is monitored continuously for 1 hour by detection of an emission wavelength 460 nm with an excitation wavelength 360 nm. Data are performed with two technical replicates.

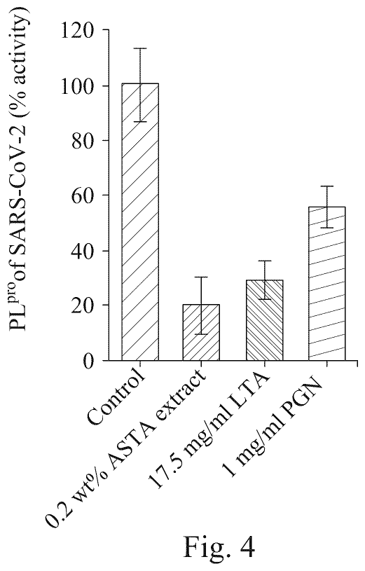

Please refer to FIG. 4, which shows inhibition of the activity of the PL^{pro} of the SARS-CoV-2 by the ASTA extract, the LTA or the PGN. In FIG. 4, the activity of the PL^{pro} of the SARS-CoV-2 in the test group treated with 1 mg/ml PGN is decreased to 60%, the activity of the PL^{pro} of the SARS-CoV-2 in the test group treated with 17.5 mg/ml LTA is decreased to 30%, and the activity of the PL^{pro} of the SARS-CoV-2 in the test group treated with 0.2 wt % ASTA extract is decreased to 20%. The results in FIG. 4 indicate that the ingredients of the nutritional composition for inhibiting survival of the RNA virus of the present disclosure can effectively reduce the activity of the PL^{pro} of the SARS-CoV-2.

4. The Inhibitory Effect on an Infection Efficiency of a SARS-CoV-2 Variant

In response to the epidemic, the nutritional composition for inhibiting survival of the RNA virus of the present disclosure can be further applied to the SARS-CoV-2 variant.

To access the inhibitory effect of the ASTA extract, the LTA, the PGN and the nutritional composition for inhibiting survival of the RNA virus of the present disclosure on a cell entry of a SARS-CoV-2 Omicron variant (hereinafter referred to as “Omicron variant”), a viral pseudo-particles (Vpp) infection assay is performed as follows. 5,000 Vero E6 cells are cultured in Dulbecco's Modified Eagle's Medium (DMEM) supplemented with 10% fetal bovine serum (FBS), 1× GlutaMAX, and 1% penicillin/streptomycin, and incubated at 37° C. and 5% CO₂. The Vpp infection assay is then performed as follows. The Vero E6 cells are seeded in a 96-well plate and cultured overnight. The next day, the Vero E6 cells are pre-incubated with 0.2 wt % ASTA extract (hereinafter referred to as “ASTA group”), 5 mg/ml LTA (hereinafter referred to as “LTA group”), 1 mg/ml PGN (hereinafter referred to as “PGN group”), 0.2 wt % ASTA extract and 5 mg/ml LTA (hereinafter referred to as “Example 2”), 0.2 wt % ASTA extract and 1 mg/ml PGN (hereinafter referred to as “Example 3”), or 0.2 wt % ASTA extract, 5 mg/ml LTA, and 1 mg/ml PGN (hereinafter referred to as “Example 4”) for 1 hour as six test groups, and the Vero ES cells pre-incubated with the DMSO at the final concentration of 5% v/v for 1 hour is as a control group. Then, the Vero E6 cells are infected with the Vpp harboring spike protein of the Omicron variant and a luciferase reporter (purchased from National RNAi Core Facility (NRC), Academia Sinica) and followed by centrifugation at 1,250×g for 30 minutes. After 24 hours incubation, a Cell Counting Kit-8 (CCK-8) assay (Dojindo Laboratories) is performed to measure a cell viability of each of the test groups or the control group. The test groups and the control group are mixed with an equal volume of ready-to-use luciferase substrate Bright-Glo Luciferase Assay System (Promega) afterward. A relative light unit (RLU) is measured immediately by the GloMax Navigator System (Promega) and normalized with the cell viability first, then the control group is set as 100% and a relative infection efficiency of each the test groups or the control group is calculated.

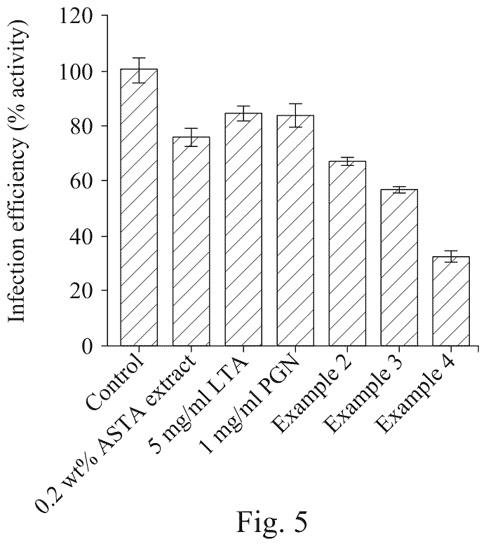

Please refer to FIG. 5, which shows the inhibitory effect on an infection efficiency of the Omicron variant of the ASTA extract, the LTA, the PGN or the nutritional composition for inhibiting survival of the RNA virus of the present disclosure. In FIG. 5, the inhibitory effect on the infection efficiency of the Omicron variant by the ASTA extract, the LTA, the PGN or the nutritional composition for inhibiting survival of the RNA virus of the present disclosure are provided herein. In the Vpp infection assay, an infection efficiency of the ASTA group, the LTA group or the PGN group is only decreased to close to 80%, respectively. However, an infection efficiency of Example 2 is decreased below 80%, and an infection efficiency of Example 3 is below 60%. Furthermore, an infection efficiency of Example 4 even falls into near 30%. The results in FIG. 5 indicate that the nutritional composition for inhibiting survival of the RNA virus of the present disclosure can effectively inhibit the cell entry of the Omicron variant, and there is a strong synergism among the ASTA extract, the LTA, and the PGN to further educe the infection efficiency of the Omicron variant. Furthermore, the results in FIG. 5 also indicate that the nutritional composition for inhibiting survival of the RNA virus of the present disclosure can effectively inhibit the infection efficiency of the SARS-CoV-2 variant. While the SARS-CoV-2 spike protein mutates so fast so as to infect the host more easily, the nutritional composition for inhibiting survival of the RNA virus of the present disclosure still can well block a way that the SARS-CoV-2 variant enters the host's cell, thereby fundamentally boosting people's capability to fight against the COVID-19 pandemic.

5. The Inhibitory Effect on the Activity of the RdRp of the SARS-CoV-2 and the Influenza A Virus

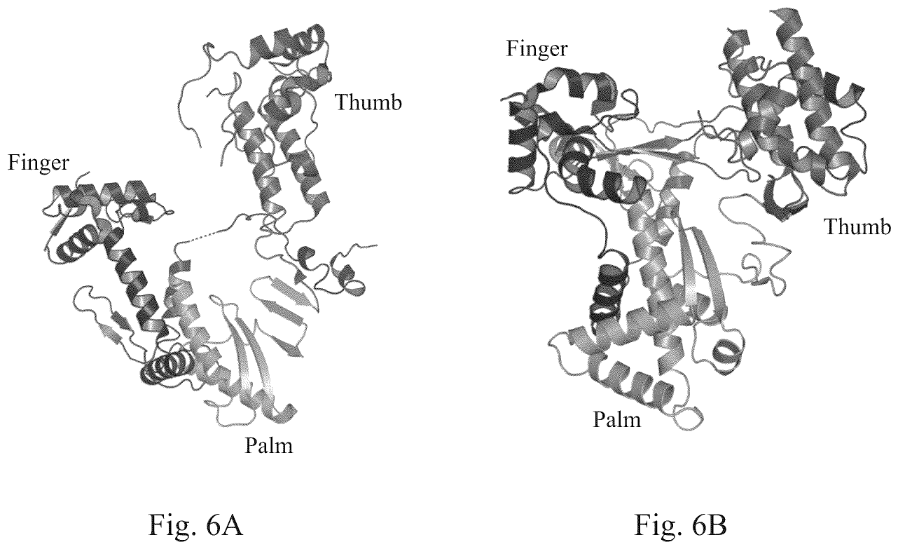

One of the most important druggable targets for the RNA virus is the RdRp. Reference is made to FIG. 6A and FIG. 6B, FIG. 6A is a schematic view showing a protein structure of the RdRp of the influenza A virus, and FIG. 6B is a schematic view showing a protein structure of the RdRp of the SARS-CoV-2. According to the comparison of structural homology in FIG. 6A and FIG. 6B, cores of the RdRp of the influenza A virus and the SARS-CoV-2 have similar 3D three-dimensional structures. Therefore, it is inferred that the nutritional composition for inhibiting survival of the RNA virus of the present disclosure has similar inhibitory effect on the RdRp of the influenza A virus and the RdRp of the SARS-CoV-2.

To access the inhibitory effect of the ASTA extract on the activity of the RdRp of the influenza A virus and the activity of the RdRp of the SARS-CoV-2, 25 μl reaction mixture containing 20.5 μl of deionized water, 2.5 μl of 10× Buffer, 0.5 μl of 50×DNA template, 0.5 μl of 50×RdRp of the influenza A virus (or 50×RdRp of the SARS-CoV-2), 0.5 μl of 50×NTPs and 0.5 μl of the ASTA extract with indicated concentration (0.4 wt %, 0.2 wt %, 0.1 wt %) or DMSO (as a control group) are incubated at 30° C. for 2 hours, respectively. The reaction mixture is then mixed with 65 μl of 1× fluorescent dye and measured the fluorescence intensity at 535 nm using the excitation wavelength at 485 nm. Data are performed with two technical replicates.

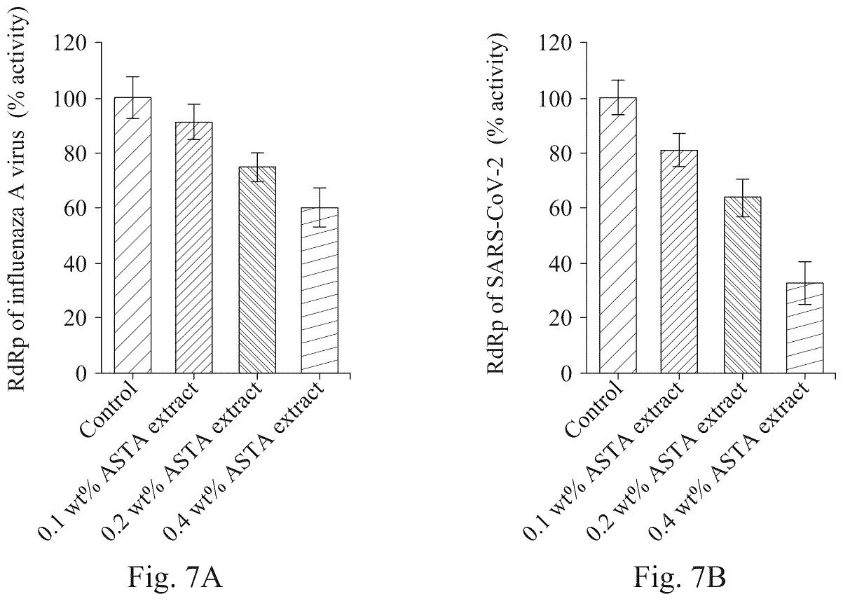

Reference is made to FIG. 7A and FIG. 7B, FIG. 7A shows inhibition of the activity of the RdRp of the influenza A virus by the ASTA extract, and FIG. 7B shows inhibition of the activity of the RdRp of the SARS-CoV-2 by the ASTA extract. In FIG. 7A, the activity of the RdRp of the influenza A virus in the test groups treated with 0.1 wt % ASTA, 0.2 wt % ASTA and 0.4 wt % ASTA are decreased to about 90%, below 80% and about 60%, respectively. In FIG. 7B, the activity of the RdRp of the SARS-CoV-2 in the test groups treated with 0.1 wt % ASTA, 0.2 wt % ASTA and 0.4 wt % ASTA are decreased to about 80%, about 60%, and below 40%, respectively. The results in FIG. 7A and FIG. 7B indicate that the ingredients of the nutritional composition for inhibiting survival of the RNA virus of the present disclosure can effectively reduce the activity of the RdRp of the influenza A virus and the activity of the RdRp of the SARS-CoV-2.

In summary, the nutritional composition for inhibiting survival of the RNA virus of the present disclosure shows excellent inhibitory effect on diverse survival aspects of the influenza A virus and the SARS-CoV-2. At a stage before the SARS-CoV-2 invades the host, the nutritional composition for inhibiting survival of the RNA virus of the present disclosure can effectively inhibit the SARS-CoV-2 spike protein to bind the ACE2 so as to disrupt an infection from the SARS-CoV-2. At a stage after the influenza A virus or the SARS-CoV-2 enters the host's body unfortunately, the nutritional composition for inhibiting survival of the RNA virus of the present disclosure can effectively decrease the activity of the RdRp of the influenza A virus or the activity of the RdRp of the SARS-CoV-2, so as to reduce viral replication. In addition, the nutritional composition for inhibiting survival of the RNA virus of the present disclosure can effectively reduce the activities of the two lethal proteases of the SARS-CoV-2, the M^{pro} and the PL^{pro}, so as to destroy a viral replication system and further block a way how the SARS-CoV-2 interferes the host's signaling pathway and a way how the SARS-CoV-2 evades the host's antiviral immune responses. Furthermore, the nutritional composition for inhibiting survival of the RNA virus of the present disclosure can be applied to deal with the SARS-CoV-2 variant. Therefore, the nutritional composition for inhibiting survival of the RNA virus of the present disclosure can effectively help people to reach a purpose of fighting against the RNA virus infection in daily life.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A nutritional composition for inhibiting survival of an RNA virus, consisting of an astaxanthin extract and at least one probiotic extract, wherein the at least one probiotic extract is selected from the group consisting of a lipoteichoic acid and a peptidoglycan.

2. The nutritional composition for inhibiting survival of the RNA virus of claim 1, wherein an effective concentration of the astaxanthin extract is equal to or larger than 0.1 wt %.

3. The nutritional composition for inhibiting survival of the RNA virus of claim 1, wherein an effective concentration of the lipoteichoic acid is equal to or larger than 1 mg/ml.

4. The nutritional composition for inhibiting survival of the RNA virus of claim 1, wherein an effective concentration of the peptidoglycan is equal to or larger than 1 mg/ml.

5. The nutritional composition for inhibiting survival of the RNA virus of claim 1, wherein the RNA virus is a severe acute respiratory syndrome coronavirus 2 (SARS-CoV-2) or an influenza A virus.

6. A method for treating an RNA virus infection, the method comprising administering an effective concentration of a nutritional composition to a subject in need thereof, wherein the nutritional composition consists of an astaxanthin extract and at least one probiotic extract, and the at least one probiotic extract is selected from the group consisting of a lipoteichoic acid and a peptidoglycan.

7. The method for treating the RNA virus infection of claim 6, wherein the nutritional composition is capable as an RNA-dependent RNA polymerase (RdRp) inhibitor.

8. The method for treating the RNA virus infection of claim 7, wherein the RNA virus infection is caused by an influenza A virus.

9. The method for treating the RNA virus infection of claim 7, wherein the RNA virus infection is caused by a severe acute respiratory syndrome coronavirus 2 (SARS-CoV-2).

10. The method for treating the RNA virus infection of claim 9, wherein the nutritional composition is capable of inhibiting a binding of a SARS-CoV-2 spike protein to an angiotensin-converting enzyme 2 (ACE2).

11. The method for treating the RNA virus infection of claim 9, wherein the nutritional composition is capable of inhibiting an activity of a main protease (M^{pro}) of the SARS-CoV-2.

12. The method for treating the RNA virus infection of claim 9, wherein the nutritional composition is capable of inhibiting an activity of a papain-like protease (PL^{pro}) of the SARS-CoV-2.

13. The method for treating the RNA virus infection of claim 6, wherein an effective concentration of the astaxanthin extract is equal to or larger than 0.1 wt %.

14. The method for treating the RNA virus infection of claim 6, wherein an effective concentration of the lipoteichoic acid is equal to or larger than 1 mg/ml.

15. The method for treating the RNA virus infection of claim 6, wherein an effective concentration of the peptidoglycan is equal to or larger than 1 mg/ml.
